(19) United States
(12) Patent Application Publication
(10) Pub. No.: US 2006/0189840 A1
(43) Pub. Date: Aug. 24, 2006

(54) Transmyocardial delivery of cardiac wall tension relief

(75) Inventors: Robert G. Walsh, Lakeville, MN (US); Timothy R. Conrad, Eden Prairie, MN (US)

Correspondence Address: FAEGRE & BENSON LLP;PATENT DOCKETING, 2200 WELLS FARGO CENTER, MINNEAPOLIS, MN, 55402, US

(73) Assignee: Acorn Cardiovascular, Inc.

(21) Appl. No.: 11/061,010

(22) Filed: Feb. 18, 2005

(51) Int. Cl. A61M 1/10; A61F 2/00

(52) U.S. Cl. 600/16; 600/37

(57) ABSTRACT

A method for treating a disease of the heart such as congestive heart failure includes forming an access opening from a heart chamber into a pericardial space defined between an epicardial surface of the heart and a pericardium opposing the epicardial surface. A cardiac support member is deployed into said pericardial space through said access opening with said cardiac support member selected to engage an epicardial surface of said heart and relieve a wall tension of said heart.

### BACKGROUND OF THE INVENTION

1. Field of the Invention

This invention pertains to a method and apparatus for providing wall tension relief to a diseased heart. More particularly, this invention pertains to a minimally invasive technique for delivery of cardiac wall tension relief.

2. Description of the Prior Art

Congestive heart disease is a progressive and debilitating illness characterized by a progressive enlargement of the heart. As the heart enlarges, the heart performs an increasing amount of work in order to pump blood with each heartbeat. In time, the heart becomes so enlarged it cannot adequately supply blood.

An afflicted patient is fatigued, unable to perform even simple exerting tasks and experiences pain and discomfort. Further, as the heart enlarges, the internal heart valves cannot adequately close. This impairs the function of the valves and further reduces the heart's ability to supply blood.

Causes of congestive heart disease are not fully known. In certain instances, congestive heart disease may result from viral infections. The heart may enlarge to such an extent that the adverse consequences of heart enlargement continues after viral infection has passed or after the pregnancy. The disease then continues its progressively debilitating course. There are numerous other causes for congestive heart failure. These include cardiomyopathy following myocardial infarction and even, in rare instances, following stress of pregnancy. Other contributors are high blood pressure and congenital pre-disposition.

Drug therapies are available for treatment of congestive heart disease. Such therapies may slow the progression of the disease but may not halt progression of the disease. In later stages of congestive heart failure, drug therapies may be without significant benefit. There is no cure for congestive heart disease and drug therapies may have adverse side effects.

Historically, the only permanent treatment for congestive heart disease has been heart transplant. Unfortunately, such a treatment is highly invasive and there are an insufficient number of hearts available for transplant.

Many new techniques have been suggested for treating congestive heart failure. Some of these techniques are in clinical study or under regulatory review in advance of regulatory approval. Examples of such techniques include those disclosed in Assignee's U.S. Pat. No. 5,702,343 issued Dec. 30, 1997; U.S. Pat. No. 6,123,662 issued Sep. 26, 2000 and U.S. Pat. No. 6,482,146 issued Nov. 19, 2002.

The Assignee's patents describe a technique for treating congestive heart failure by placing a cardiac support device in the form of a jacket around the heart. In certain specific embodiments, the jacket is a knit of polyester material which surrounds the heart providing a resistance to progressive diastolic expansion. Other described materials include metal such as stainless steel. In certain aspects the knit sides and open cell sides are selected to minimize or control fibrosis. It is believed that such resistance decreases wall tension on the heart and permits a diseased heart to beneficially remodel.

Assignee's U.S. Pat. No. 6,730,016 issued May 4, 2004 describes a jacket with a non-adherent lining or coating. In certain embodiments, the coating is in specific locations (for example, over surface-lying cardiac blood vessels). Assignee's U.S. Pat. No. 6,425,856 issued Jul. 30, 2002 describes a cardiac jacket with therapeutic agents incorporated on the jacket for providing additional therapy to the heart. The '856 patent also describes a jacket made of bio-resorbable material. Assignee's U.S. Pat. No. 6,572,533 issued Jun. 3, 2003 describes a treatment on the left ventricle side of the heart only. Assignee's U.S. patent application Ser. No. 10/165504 filed Jun. 7, 2002 and published Dec. 12, 2003 as Publication No. 2003-0229265 A1 teaches a highly compliant cardiac jacket. Assignee's U.S. patent application Ser. Nos. 10959888 filed Oct. 5, 2004 describes cardiac wall tension relief with fibrosis agents and other drug treatments including treatments placed on the pericardium or in the space between the pericardium and the heart.

Other examples of wall tension relief are disclosed in U.S. Pat. No. 6,059,715 issued May 9, 2000 (assigned to Myocor Inc.). The '715 patent describes various geometries for applying force to external surfaces of the heart to reduce wall tension on the heart. U.S. Pat. No. 6,508,756 issued Jan. 21, 2003 (assigned to Abiomed Inc.) describes a passive cardiac assistance device. U.S. Pat. No. 6,682,474 dated Jan. 27, 2004 (assigned to Paracor Surgical Inc.) describes an expandable cardiac harness for treating congestive heart failure. The '474 patent describes a harness made of nitinol.

In addition to mechanical devices for surrounding the heart, congestive heart failure is also being investigated for treatment through techniques for cardiac pacing of the heart (particularly, so-called “bi-ventricular pacing”). Pacing can also be done in conjunction with a cardiac support device as disclosed in U.S. Pat. Nos. 6,076,013 and 6,587,734. Defibrillating treatments are also possible with a cardiac support device as disclosed in U.S. Pat. No. 6,370,429.

Treatments for wall tension relief are very promising. However, these treatments typically involve a surgical access to the heart. The surgical access may include a full sternotomy or a less invasive surgical access such as a port access between ribs or below the sternum. A catheter-based delivery of a heart assist device has been suggested in U.S. Pat. No. 6,808,483 to Ortiz et al. dated Oct. 26, 2004. FIGS. 12-13B show what is referred to as a “partially catheter-based implantation” and FIGS. 14A and 14B show what is referred to as a “completely catheter-based” system.

Notwithstanding the significant amount of effort being placed on developing treatments for congestive heart failure, additional novel treatments are needed to improve the treatment of congestive heart failure and complications related to dilated cardiomyopathy including valvular dysfunction. It is an object of the present invention to provide an improved method for providing wall tension relief to a heart in a less invasive procedure. By providing wall tension relief through a minimally invasive procedure, a less traumatic procedure can be delivered. This can enlarge the potential patient population by permitting a therapy for those patients who cannot tolerate surgical risks associated with more invasive procedures as well as permitting procedures to be done by a wider variety of health care providers including interventional cardiologists and radiologists.

### SUMMARY OF THE INVENTION

According to a preferred embodiment of the present invention, a method is disclosed for treating a disease of the heart such as congestive heart failure. The method includes forming an access opening from a heart chamber into a pericardial space defined between an epicardial surface of the heart and a pericardium opposing the epicardial surface. A cardiac support member is deployed into said pericardial space through said access opening with said cardiac support member selected to engage an epicardial surface of said heart and relieve wall tension of said heart. In one embodiment, a guide member such as a guide wire is admitted into said pericardial space through the access opening and positioned in a desired disposition for the subsequent placement of the cardiac support member. The deployment of the cardiac support member includes advancing the cardiac support member to the desired position after the deployment of the guide member with the guide member guiding the cardiac support member to the desired position. In another embodiment, an everting member is deployed through the access opening. The everting member may be left in place as the cardiac support member or be used as a guide member for subsequent deployment of another cardiac support member.

### BRIEF DESCRIPTION OF THE DRAWINGS

FIG. 1 is a schematic cross-sectional view of a human heart surrounded by a pericardium and illustrating various anatomical features;

FIG. 2 is the view of FIG. 1 showing formation of an access opening through a wall of a right atrium and into a pericardial space and showing partial deployment of a guide wire into the pericardial space;

FIG. 2A is the view of FIG. 2 showing formation of an access opening through a wall of a left atrium and into a pericardial space by passing a catheter through a septal wall between the atria;

FIG. 3 is the view of FIG. 2 with only the pericardium shown in cross-section and showing a guide wire fully deployed in a spiral pattern around the heart and within the pericardial space;

FIG. 4 is the view of FIG. 3 showing a cardiac support device deployed into the pericardial space over the guide wire of FIG. 3 and positioned in a spiral pattern over the heart;

FIG. 4A is a cross-sectional view of the cardiac support device of FIG. 4 over a guide wire an illustrating an embodiment of a cardiac support device with a circular cross-section;

FIG. 4B is a view similar to that of FIG. 4A and illustrating an alternative embodiment with a cardiac support device having a flattened oval cross-section;

FIG. 4C is a view similar to that of FIG. 4A and illustrating an alternative embodiment with a cardiac support device having a circular cross-section with stabilizing out-rigging;

FIG. 5 is the view of FIG. 4 showing an alternative embodiment with the cardiac support device shown as two separate rings surrounding the heart in the pericardial space;

FIG. 6 is a view taken generally along line 6-6 of FIG. 5 and showing a cardiac support device encircling the heart;

FIG. 7 is a view similar to that of FIG. 6 and showing an alternative embodiment with a cardiac support device only partially encircling the heart;

FIG. 8 is a cross-section view of a catheter penetrating a right atrial wall into a pericardial space and showing initial deployment of an everting member into the pericardial space;

FIG. 9 is the view of FIG. 8 showing further deployment of the balloon into the pericardial space;

FIG. 10 is a transverse cross-section view of a heart and showing a cardiac support device on an epicardial surface of the heart and showing a first embodiment of attachment of the device to the epicardium;

FIG. 11 is a view similar to that of FIG. 10 and showing a second embodiment of attachment of the device to the epicardium;

FIG. 12 is a cross sectional view of the everting balloon of FIGS. 8 and 9 and illustrating use of the balloon to guide a separate cardiac support device through a lumen of the balloon;

FIG. 13 is a view similar to that of FIG. 12 and showing use of the balloon to guide a separate cardiac support device around an outer surface of the balloon;

FIG. 14 is a view similar to that of FIG. 4 and showing spacers between the heart and the pericardium as the cardiac support device;

FIG. 15 is a view taken generally along line 15-15 of FIG. 14 and showing spacers on diametrically opposite sides of the heart;

FIG. 16 is the view of FIG. 15 and illustrating a modified embodiment with elongated spacers;

FIG. 17 is a view similar to FIG. 3 and showing deployment of an everting cardiac harness; and

FIG. 18 is a longitudinal cross-sectional view of a distal end of an everting balloon device.

### DETAILED DESCRIPTION OF THE PREFERRED EMBODIMENT

With reference to the various drawing figures in which identical elements are numbered identically throughout, a description of the preferred embodiment of the present invention will now be provided. Assignee's aforementioned U.S. Pat. Nos. 5,702,343; 6,123,662; 6,482,146; 6,730,016; 6,425,856 and 6,572,533 and U.S. Patent Application Publication No. 2003-0229265 A1 are incorporated herein by references those set forth in full. Further, the aforementioned U.S. Pat. Nos. 6,059,715; 6,508,756 and 6,682,474 are incorporated herein by reference as though set forth in full.

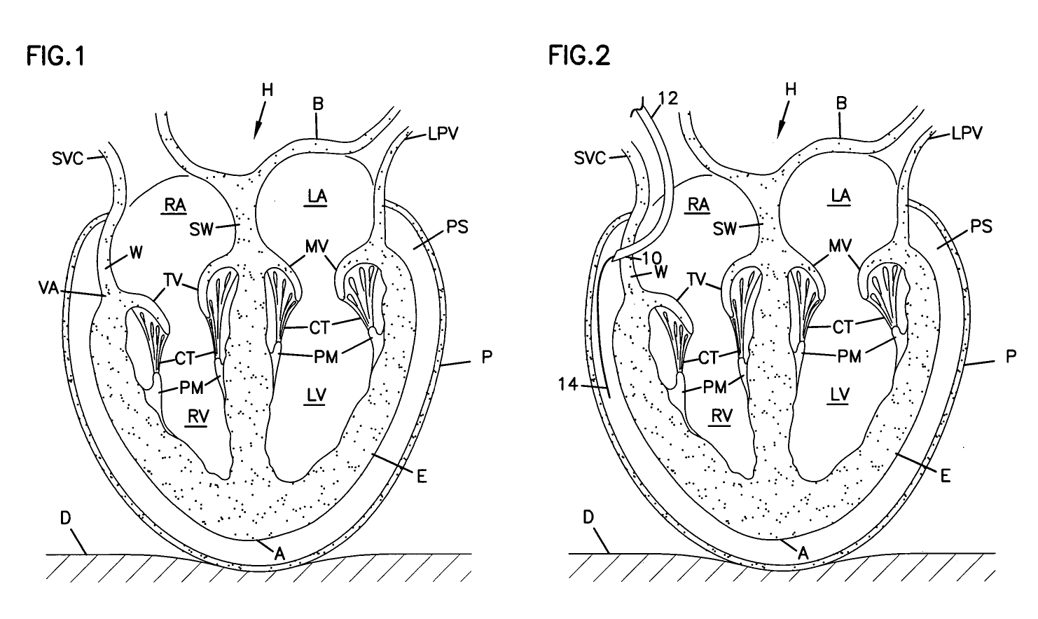

With initial reference to FIG. 1, a human heart H is schematically shown in cross-section. The heart H has a length from an apex A to a base B. FIG. 1 illustrates a left ventricle LV, a right ventricle RV, a left atrium LA and a right atrium RA. The atria LA, RA are separated from the ventricles LV, RV by a valvular annulus VA region in the region of heart valves including the tricuspid valve TV and the mitral valve MV.

Muscular extensions (referred to as papillary muscles PM) within the ventricles LV, RV are attached to the valves TV, MV by cordae tendineae CT to control function of the valves TV, MV. A plurality of veins enter the right atrium RA. Only the superior vena cava SVC is shown for ease of illustration. A plurality of pulmonary veins enter the left atrium LA. For ease of illustration only the left pulmonary vein LPV is shown. The heart H has a pericardium P as a sack surrounding the heart H with an apex attached to the diaphragm D. The space between the pericardium P and the epicardial surface E of the heart H is referred to as the pericardial space PS.

In a preferred embodiment, the present invention uses a transatrial approach to the pericardial space PS. Transatrial access to the pericardial space PS is known for obtaining diagnostic sampling or to administer therapeutic agents (such as pharmacologic or cellular agents). In a transatrial access, a hollow needle is passed into the right atrium RA through a vein such as the superior vena cava SVC. The needle is urged through the right atrial wall (or the right atrial appendage) to form a hole through the atrial wall. The hole thereby connects the interior right atrium RA to the pericardial space PS. A guide wire is passed through the hollow needle into the pericardial space PS and the needle is removed. A catheter can then be guided over the guide wire into the pericardial space PS. The guide wire is then removed. Collection of pericardial fluid for diagnostic purposes is performed through the catheter. This procedure is described in greater detail in Verrier et al. “Transatrial Access to the Normal Pericardial Space”, Circulation, page 2331-2333 (Dec. 8, 1998). While a needle is disclosed in Verrier et al. to form an access hole, other options are available (e.g., forming the hole through the atrial wall with a hollow guide wire without need for a catheter).

Throughout the present application, access to the pericardial space PS will be described with reference to a transatrial access as described in the Verrier, et al. article. It will be appreciated (and as illustrated in FIG. 2A) that access need not be from the right atrium RA that could be through the left atrium LA. A needle and catheter 12 could be passed through a transit hole 10″ formed through the septal wall SW dividing the right atrium RA and the left atrium LA. The catheter 12 can then be passed through an access opening 10′ formed through the left atrial wall W″ into the pericardial space PS.

It is presently believed that passing a needle through the right atrial wall is most preferred due to a low pressure differential between the right atrium RA and the pericardial space PS minimizing risk of blood loss into the pericardial space PS. In the Verrier et al. article, a four French (approximately 1.3 mm diameter) aspiration catheter was passed into the pericardial space from the right atrium and withdrawn without need for a suture or the like to repair the puncture hole. In the event a larger catheter is to be passed through the right atrial wall, a purse string suture could be provided at the point of puncture to aid in healing the puncture wound. Other techniques for closing such a wound include a hemostatic plug.

Guide Wire Deployment

According to the present invention (and as shown in FIG. 2), an access hole 10 is formed through the outer wall W of the right atrium RA into the pericardial space PS in a manner as described in the Verrier et al article with a hollow catheter 12 passed through the hole 10 with a distal end residing within the pericardial space PS. A guide wire 14 is passed through the catheter and deployed in the pericardial space PS. In later embodiments, alternative deployment of a cardiac support device will be described (e.g., with reference to an everting balloon).

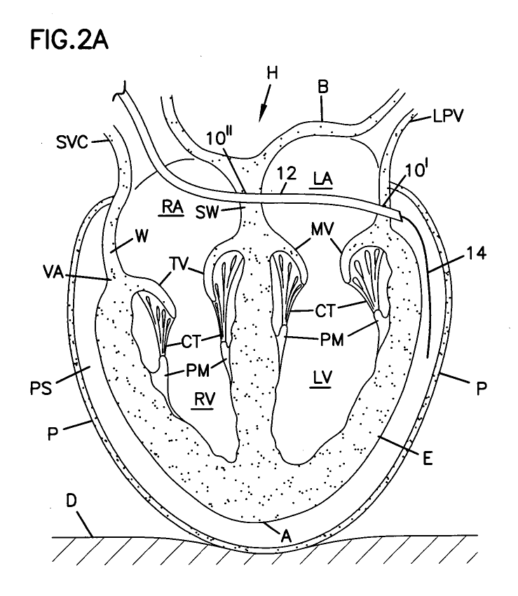

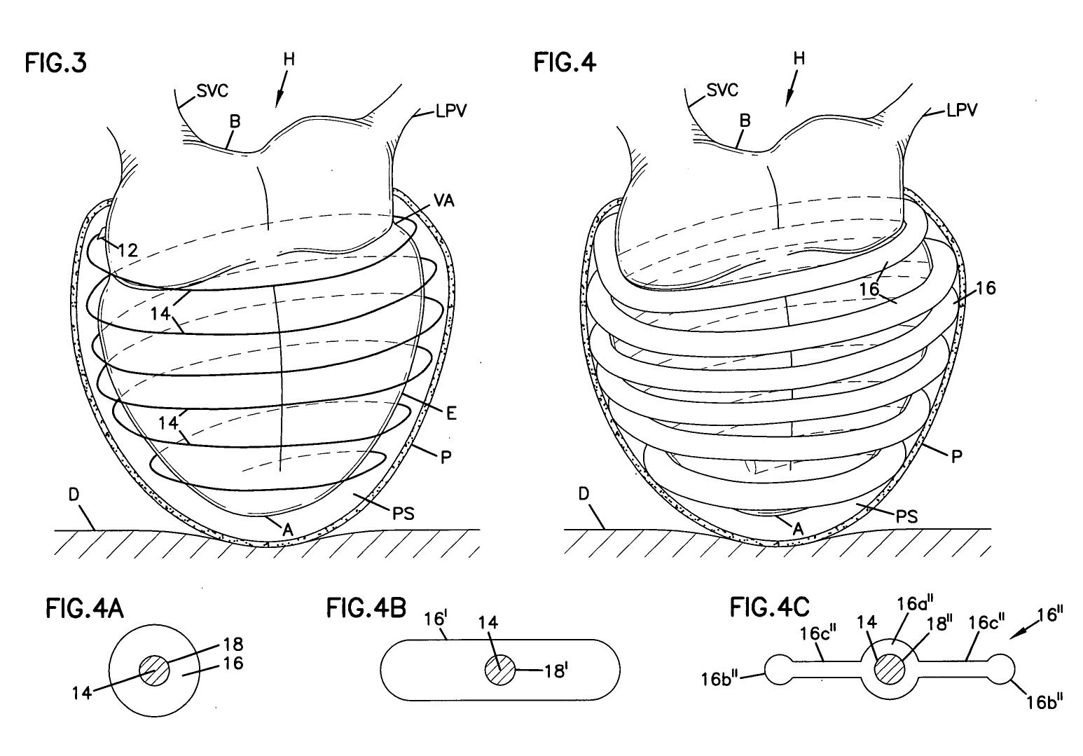

As illustrated in FIG. 3, the guide wire 14 is advanced through the pericardial space PS to be deployed around the heart H in any desired pattern. In FIG. 3, the desired pattern is shown as a spiral positioning of the guide wire 14 around the heart H between the epicardial surface E and the pericardium P and extending from the valvular annulus VA toward the apex A of the heart H. Accordingly, the guide wire 14 will surround the valvular annulus VA as well as surrounding the region of the ventricles LV, RV overlying the papillary muscles PM.

The guide wire 14 may be formed of any suitable material of adequate flexibility to surround the heart H in the pericardial space PS. It will be appreciated that guide wires are well known. The guide wire 14 may be formed of a highly elastic material or a shape-memory material such as nitinol to assist in ease of its placement. Alternatively, the guide wire 14 may have a magnetic distal tip to permit the guide wire to be magnetically manipulated by movement of a magnetic field around the patient to draw the guide wire 14 around the heart in any desired pattern such as the spiral pattern shown in FIG. 3 or in a circular pattern as will be later described. Other techniques for directing a guide wire in the pericardial space PS include passing a tool through the pericardium to grasp and direct the guide wire. Such a tool can be introduced through a port access such as access through an intercostals space or sub-xyphoid.

Once the guide wire 14 is in place within the pericardial space PS in the desired pattern, a passive constraint device 16 (also referred to as a cardiac support device) can be deployed. For use with a guide wire assisted deployment, the cardiac support device 16 is a flexible elongated member having a lumen 18 (FIG. 4A) sized to slidably receive the guide wire 14). As shown in FIG. 4, after fully sliding the cardiac support device 16 over the guide wire 14, the device 16 resides completely in the pericardial space PS and surrounds the heart H in the desired pattern. The guide wire 14 and catheter 12 can then be withdrawn and the access opening 10 can be repaired (if necessary or left to heal by thrombus formation in the hole 10). The cardiac support device 16 may be secured in place through any suitable means. Later, optional fixation methods will be described.

In FIGS. 4 and 4A, the cardiac support device 16 is a tubular structure of circular cross-section with the lumen 18 axially positioned within the cardiac support device 16. Other geometries are possible. For example, FIG. 4B shows a cardiac constraint device 16′ with a flattened oval cross-section. This geometry presents a lower profile and would more easily be advanced through the pericardial space PS. FIG. 4C illustrates a cardiac support device 16″ with a smaller diameter central portion 16a” (containing the lumen 18” for the guide wire 14) and having parallel spaced-apart out-riders 16b” connected to the central portion 16a” by flat extensions 16c”. The geometry of FIG. 4C presents a low profile in the pericardial space PS. The wide dimension (measured between the out-riders 16b”) and flat geometry provides a more stable positioning on the epicardial surface E.

The passive constraint device 16 is selected to be any material which, when in place around the heart H, provides wall tension relief by presenting a resistance to diastolic expansion as described in the aforementioned patents. The passive constraint device 16 may include, in whole or part, nitinol or other shape memory material amenable to deployment as a helix around the heart. The material may be formed as a solid material or a weave or knit of strands of material to enhance flexibility. The passive constraint device 16 can include, in whole or part, a polymer such as polyester or the like to promote fibrotic encapsulation of the device 16 against the epicardial surface E thus stabilizing the position of the device 16 around the heart H. The device 16 could also be a laminate of a polymer and a metal (such as nitinol or stainless steel in combination with polyester). In a laminate construction, the metal can provide shape stability and the polymer provides desired host response characteristics (such as fibrotic encapsulation). Also, the device 16 could have a polymer or metallic stiffening member disposed within the device 16 to help in placement of the device 16 which can then either be removed or left in place as part of the passive constraint device 16.

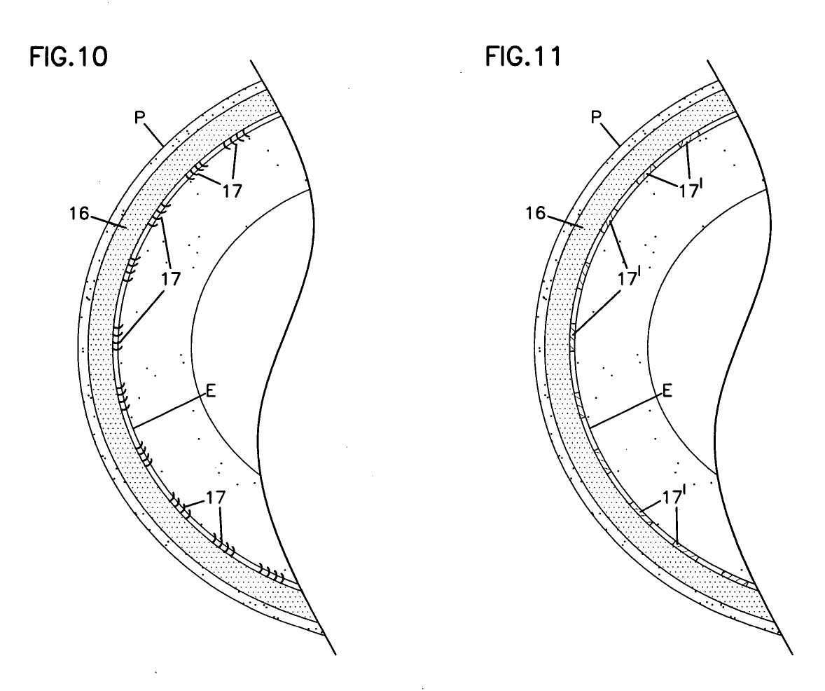

A cardiac support device 16 may have mechanisms for attachment along its length to secure the device 16 in place on the epicardial surface E. The mechanisms for attachment can be positioned at various points along the length or at its ends only to stabilize positioning of the device 16 on the epicardial surface E. Such a mechanism for attachment could include adhesives, splines or tangs for bonding to the epicardial surface. FIG. 10 illustrates barbs 17 secured to the device 16 for penetration into the muscle of the heart H at the epicardial surface E. FIG. 11 illustrates an alternative attachment mechanism includes spaced patches 17′ which may be an adhesive or a patch of material (e.g., polyester felt) to promote fibrosis or tissue in-growth. An example of mechanisms for attachment to an epicardial surface are disclosed and described in U.S. Pat. No. 6,846,296 to Milbocker et al. dated Jan. 25, 2005 (incorporated herein by reference).

Everting Member Deployment

Previously mentioned as an alternative to a guide wire, an everting balloon may be passed through the access opening 10 in the right atrial wall. Examples of everting balloons for passage through tortuous anatomical pathways are shown in U.S. Pat. Nos. 5,630,797, 5,389,089 and 5,374,247 (all incorporated herein by reference) used for providing access to an obstructed fallopian tube in gynecologic treatments. An everting balloon has the added benefit that it need not be pushed through a pericardial space. The balloon rolls out thereby encountering less friction resistance to deployment and minimizing sliding trauma against opposing tissue.

FIG. 8 illustrates an everting balloon 20 being deployed from a distal end of a catheter 21 into the pericardial space PS. Further deployment results in the balloon 20 being advanced into the pericardial space PS as illustrated in FIG. 9. Balloons may be preformed with a desired shape so that as the balloon is deployed, it is biased to surround the heart in a desired pattern (such as the spiral pattern of the device in FIG. 4 or a circular deployment). The balloon 20 may be used as a deployment aid of a permanent passive device or the balloon 20 itself may be left in place as a passive constraint device as will be described.

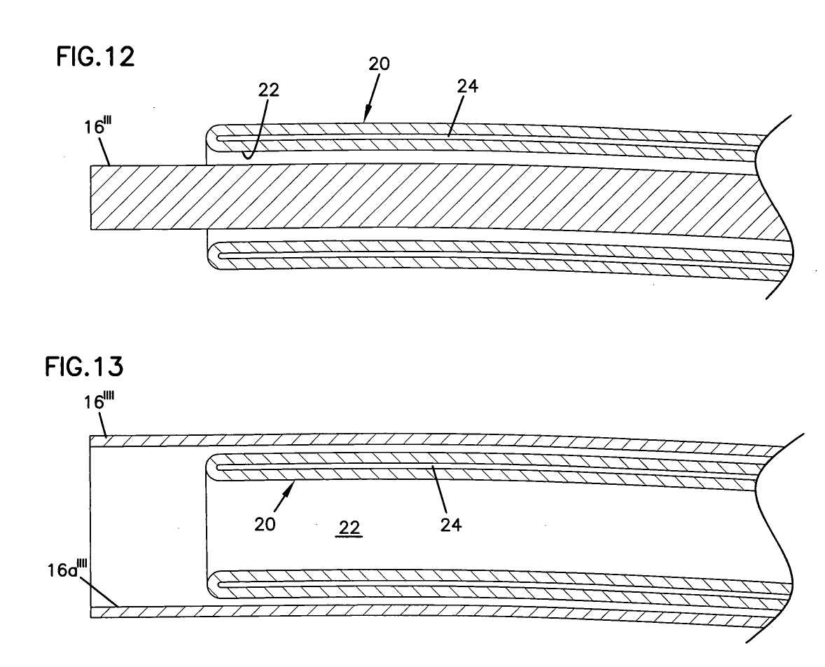

The balloon 20 has a fluid chamber 24 (best shown in FIGS. 12 and 13) to receive a fluid to deploy the balloon 20. The balloon 20 is a hollow structure having an internal lumen 22. As a result, a cardiac support device 16′″ may be passed through the lumen 22 as illustrated in FIG. 12. FIG. 13 illustrates a support device 16″″ which has a lumen 16a″″ so that it is passed over the balloon 20. Using a balloon 20 as a guide member, the balloon 20 is deployed in the pericardial space PS to the desired pattern around the heart H. The balloon 20 then guides the delivery of the cardiac support device (16′″, 16″″) to the desired pattern. The balloon 20 is then removed leaving the cardiac support device in place surrounding the heart.

As mentioned, a separate cardiac support device need not be placed using an everting balloon as a guide member. Instead, the everting balloon 20 may be left in place and maintained with an internal pressure in the chamber 24 (by saline injection) to provide adequate constraint on the heart. Rigidity in position can be adjusted based on the balloon pressure. The balloon 20 may be released from the catheter with any sealing valve sealing a proximal end of the balloon 20 upon its release. Alternatively, the balloon 20 may be inflated by saline injected into the chamber 24 through a needle. The material of the balloon 20 can include a self-sealing membrane to maintain a seal following removal of the needle

The balloon may be pre-cast such that when pressurized it assumes the desired shape and geometry (for example, helical spiral or circular) of the implanted passive constraint device. As an alternative, the balloon serves as a surface for contact with the epicardium with an internal member (nitinol or other shape memory metal) providing shape and geometry to the passive constraint device.

Pericardial Assist

In previously described embodiments, the support device provides resistant to diastolic expansion by reason of having a material surrounding the heart. The material of the device may be inelastic or elastic to accommodate diastolic expansion with an opposing load.

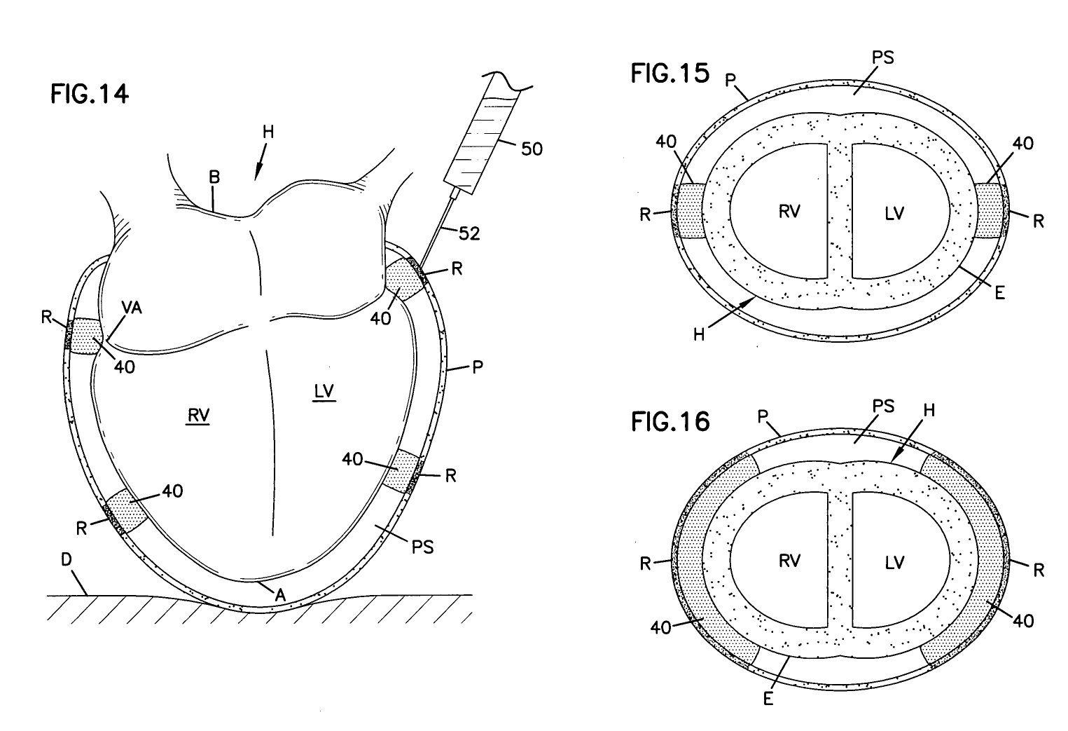

In addition to the above, the pericardium P can be used to cooperate with a spacer to resist diastolic expansion. In FIGS. 14-16, a spacer material 40 is placed between the pericardium P and the epicardium E. The spacer material 40 can be a polymer or metal as previously described. The material 40 is deployed from a catheter into the pericardial space PS through transatrial delivery as described above. It can be placed opposing the valvular annulus VA or opposing the ventricles RV, LV in the region of the papillary muscles PM.

As the heart H expands, the spacer material 40 transmits the expansion forces directly to the pericardium P which can present a load resisting such expansion. The resistance of the pericardium P to expansion can be enhanced. For example, the pericardium P can be treated with a stiffening agent to stiffen the tissue of the pericardium P and resist its tendency to accommodate cardiac expansion. For example, in the region R of the pericardium P opposing the spacers 40, the pericardium P can be treated by injection of a stiffening agent 50 from a needle 52. An example of such an agent is glutaraldehyde. As a consequence, the stiffened region R of the pericardium P cooperates with the spacers 40 to resist diastolic expansion of the heart H. Alternatively, a stiffening agent can be applied to the exterior outer surface (i.e., opposing the pericardium P) of the spacers 40 to stiffen the pericardium P without exposing the stiffening agent to the heart H or the pericardial space PS.

Comparison of FIGS. 15 and 16 illustrate how the amount of spacer material 40 can be varied to alter the amount of area of the heart covered by the treatment.

Apical Delivery of Cardiac Support Device

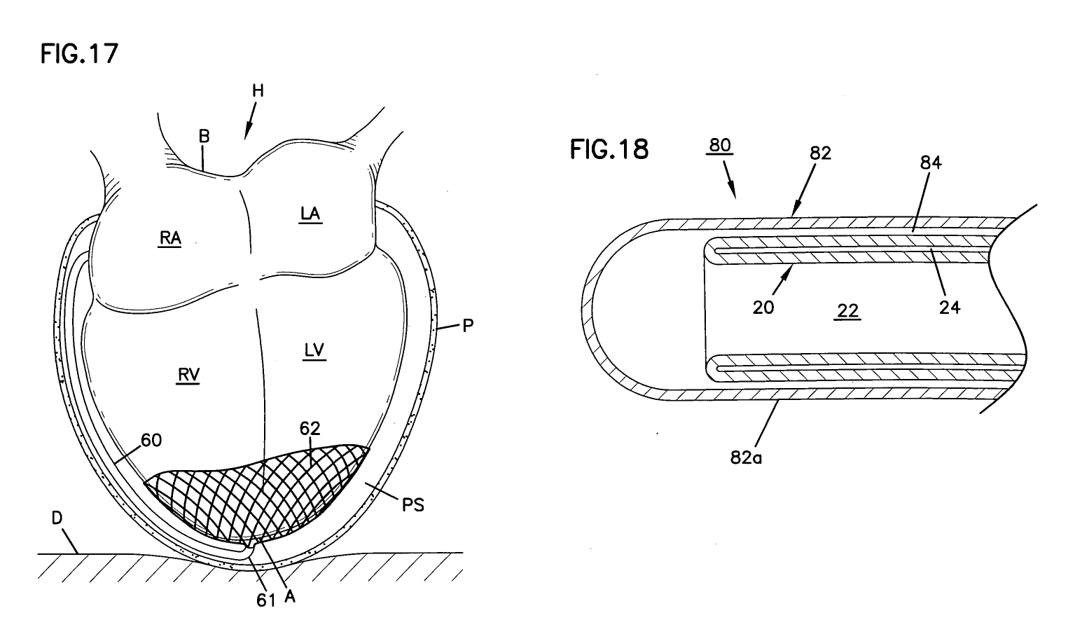

FIG. 17 illustrates placing a catheter 60 transatrially with a distal tip 61 of the catheter 60 located between the pericardium P and the apex A of the heart H. The distal tip 61 is positioned pointing toward the apex A. So positioned, an everting cardiac harness 62 (such as that described in U.S. Pat. No. 6,682,474) can be ejected from the tip 61. The pericardium P and underlying anatomical structures, such as the diaphragm D, support the catheter 60 in ejecting the everting harness 62 from the catheter 60 and onto the epicardial surface E of the heart H in the pericardial space PS.

Multiple Design Options

Multiple design options are possible with the present invention. For example, the passive constraint device 16 can be a free-floating device retained within the pericardial space PS. The interior surface of the pericardium P can help guide the passive constraint device 16 to the proper location within the pericardial space PS. The passive constraint device 16 is prevented from significant longitudinal movement by the pericardium P at either the pericardial inflection point near the cardiac base or at the apex. Alternatively, an attachment mechanism as previously described may be applied at desired locations.

If an everting balloon 20 is used as an integral part of the passive constraint device, it is released as in inflated member from the introduction catheter 21. In such a case, the proximal end of the balloon is valved as described with a closure device for inflating, closing and separating the balloon 20 from the device introduction catheter 21. The proximal end of the balloon 20 may be provided with a valve fitting to set in place and pressure within the balloon which is reversibly attached to a pressurization catheter component when the everting balloon is pressurized. Alternatively, the balloon 20 could include a self-sealing polymer and the inflation device could be a simple needle placed within the chamber 24 of the balloon 20 and removal of the needle results in self-sealing of the balloon 20. The needle placement within the balloon 20 permits injection of saline or other fluid into the chamber 24 for pressurization.

Use with Prior Surgery Patients

As thus described, the procedure is performed on an intact pericardium P and the pericardium P is left intact. The method and apparatus of the present invention is suitable for patients who have never had cardiac surgery as well as other patients who have had previous cardiac surgery. In the latter case, the pericardium would not be intact, and surgical adhesions might make it difficult to deploy the passive constraint device. To accommodate this, the device can be adapted to incorporate a directional endoscope. Such endoscope could be used to monitor progress during deployment of the everting balloon and/or passive constraint device, and help in passing around areas of adhesion involving the epicardial surface. Alternatively, the endoscope can aid in passing the everting balloon and/or passive constraint device through such areas of adhesion, particularly if they are not too extensive or rigid or mature. The endoscope can be combined with methods for transatrial access, involving passage of a needle across the adhesion or pericardial remnant, followed by placing a guide-wire, followed by advancing the endoscope or passive constraint device across the obstruction. An endoscope having an ability to steer also helps in positioning the everting balloon or passive constraint device for situations involving an intact pericardium as well.

Delivery of Bioactive Agents

The passive constraint device (e.g., 16, 16′, 16″, 16′″, 16″″, 16a, 16b, 20, 40, 62) described in this disclosure can be adapted for local delivery of bioactive agents. As that term is used herein, a bioactive agent includes one or more of the following: low molecular weight pharmaceuticals/drugs, genes, gene products such as proteins or messenger RNAs, and cells. As such, the materials employed in fabricating the passive constraint device can be adapted to incorporate the various agents, either directly in the passive constraint itself, or within coatings deployed on the surface of the passive constraint device. Therefore, bioactive agent-containing polymer coatings can be deposited upon the surface of the everting balloon, or a metallic or polymeric member implanted into position around the heart as a results of deployment of an everting balloon. The bioactive agent-containing coating can also be applied to fabric or other polymeric or metallic members affixed to the everting balloon.

Use in Pacing and Electrical Diagnostics

In another iteration of the passive constraint device, the device (e.g., 16, 16′, 16″, 16′″, 16″″, 16a, 16b, 20, 40, 62) can be formed of electrically conductive material or have multiple individual electrodes placed along the length of the device. Such a device can be used to map electrical conductivity of the heart, as well as serve as a multiple electrode array for stimulating the heart in a multi-site pacing method. The number of electrodes can vary from one to 10 or more. The electrodes can be connected to a controller to allow switching of electrodes for rapid mapping/diagnostics and acute testing of various electrode locations for achieving optimal pacing characteristics (based on hemodynamics). In a presently preferred embodiment, the combination of electrodes consists of up to 4 electrodes total, and stimulation can either be simultaneous, or sequential in order to achieve optimized synchronous ventricular contraction and optimized cardiac hemodynamic performance. An implantable pulse generator can then be attached to the leads necessary to achieve the desired optimal hemodynamics in chronic use.

Alternative Positioning Options

The deployed passive constraint device can consist of a single complete or partial circumferential deployment around the heart, and be positioned at or near the atrioventricular groove. For example, FIG. 5 shows a first cardiac support device 16a at the valvular annulus VA and a second cardiac support device 16b surrounding the ventricles LV, RV in the region of the papillary muscles PM. Either or both positions can be used. These positions serve to treat valvular dysfunction as well as congestive heart failure.

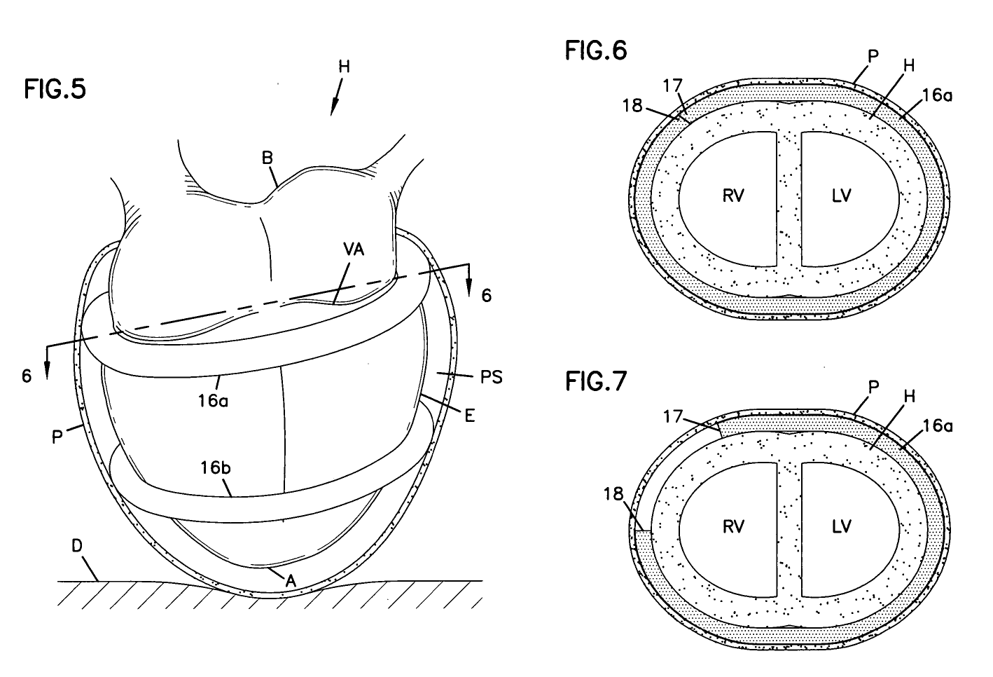

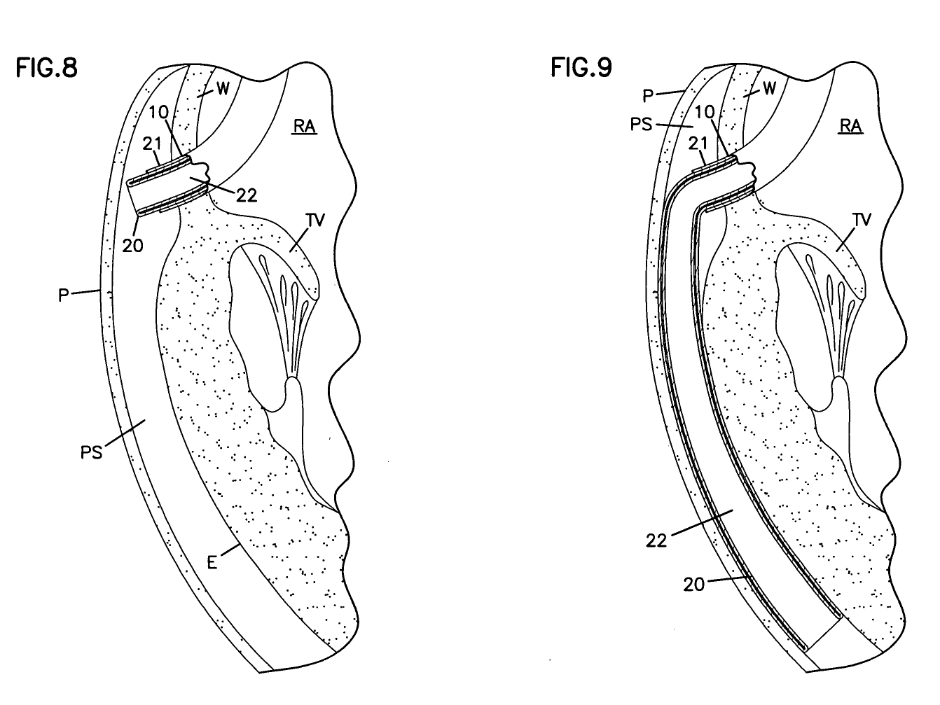

As shown in FIG. 6, the cardiac support device 16a completely surrounds the heart H. In FIG. 7, the device 16a only partially surrounds the heart H but preferably is sized to cover at least the portion of the valvular annulus VA surrounding the mitral valve MV.

A single support device in the valvular annulus VA applies inward pressure upon the mitral valve annulus VA in order to decrease the extent of mitral regurgitation. The device acts as a mitral annulus support device since it does not support the entire heart H. Surrounding the ventricles LV, RV in the region of the papillary muscles PM also supports valvular function.

In FIG. 6, the support device 16a completely encircles the heart H in one rotation, and then connects at ends 17, 18 to form a continuous ring. Such a ring can have an adjustable diameter allowing fine-tuning of compression on, or adjacent to, the valvular annulus VA. Such connection may be any suitable connection method such as tabs, buttons, hook-and-loop fasteners (e.g., Velcro™ brand fasteners), bayonet, zip tie, screw, latch, lock and key, hook, or buckle. Alternatively, inflation (in the case of a balloon-containing passive constraint device) can adjustably provide inward compressive force of the deployed device, thereby reducing size of the mitral annulus and reducing the degree of mitral regurgitation.

An alternative mechanism of introducing mechanical stress into a support device is based on the principles adapted from a bimetallic thermometer or thermostat. The support device is fabricated from two separate parallel lengths of metal of roughly equivalent length, which have contact with each other, and which can slide in reference to each other. Placing tension on one of these lengths of metal, via the introduction catheter 12 causes the entire device to bend, thus applying compressive force to the heart H. Such force can be directed primarily along the atrioventricular plane to promote reduction in the mitral annulus size (mitral annulus support device), or more globally upon a portion or substantially all of the cardiac surface between the base B and the apex A (passive constraint device). Various systems could be used to make the tension permanent in the device, including a ratchet system involving the two pieces of metal. Other options are as mentioned earlier and include: tabs, buttons, hook-and-loop fasteners, bayonet, zip tie, screw, latch, lock and key, hook, or buckle connectors.

The tensioning mechanism can be designed to allow tightening or loosening of tension, and thus on the amount of compressive force generated by the device. The tensioning mechanism can be amenable to adjustment over time following deployment of the device, by reintroduction of a suitable catheter to make contact with the implanted support device. Such adjustment could be used to increase or decrease the compressive force directed towards the mitral annulus (in the case of a support device surrounding the valvular annulus VA), or directed generally towards the heart (in the case of a cardiac passive constraint device surrounding the heart H).

Active Constraint Device

The balloon 20 can be attached chronically to a separate implanted device that cyclically pressurizes and depressurizes the balloon in synchronization with cardiac contraction. In such a system, the constraint device would serve as an active constraint, assisting the heart to eject blood into the circulation during cardiac systole (and possibly assisting the ventricles in filling during cardiac diastole). Such a device for pressurizing and depressurizing the constraint device can make connection with the constraint device at one end or the other, at both ends, or at various points along the length of the constraint device, in order to facilitate rapid pressurization and depressurization. It is presumed that the constraint device would be liquid filled (preferably, saline).

Use of Resorbable Materials

Resorbable materials can be used to construct the everting balloon, should the desire be to incorporate the balloon into a passive constraint device. More preferably, the metallic or polymeric support member introduced through the everting balloon (as described above) would be fabricated from bioresorbable material, and would represent the passive constraint device remaining in contact with the epicardium after the other catheter-based components are withdrawn. There are numerous possible choices for the bioresorbable passive constraint device, including polyglycolic acid or polylactic acid, or a mixed composition of these polymers.

Injected Material

The cardiac constraining device needs not be formed for solid materials. Instead, a catheter can be inserted transatrially into the pericardial space S inject a substance into the space PS to form the cardiac support device in situ. The passive constraint device can consist of a biocompatible polymer (either permanent or biodegradable) which is injected from catheter 12 into the pericardial space PS, and then solidified after contact with the epicardial surface. An injectable material could include a gel (such as a gel based on hyaluronic acid, chondroitin sulfate, collagen, or a mixture of these materials). Such a device can be an especially suitable application for therapy of patients shortly after acute myocardial infarctions, in which case, the solidified polymer/gel composition would tend to resist chronic dilation/remodeling of the cardiac ventricles, and/or aneurysm formation involving the ventricular wall.

Such prevention can be temporary to accommodate the natural healing process after the infarction event. In such case, a biodegradable polymer/gel composition is preferred provided that degradation of the material does not impose an undue or undesirable inflammation process which could interfere with cardiac healing or function.

Double Balloon Cardiac Support Device

In still an additional iteration of the everting balloon approach to a passive constraint device, a double balloon can be used in which a porous outer balloon would be mounted over the everting balloon. FIG. 18 illustrates a longitudinal cross-section of a distal end of such a double balloon 80. The everting balloon 20 (with lumen 22 and fill chamber 24) is the inner balloon. An outer balloon 82 surround the inner balloon 20). In a preferred embodiment, the outer balloon 80 is porous and the inner balloon 20 is not porous.

Appropriate ports within the delivery catheter can allow delivery of drug solution to the space 84 between the outer porous balloon 82 and the non-porous everting inner balloon 20. Pressure would then be used to deliver bioactive agent solution across the porous balloon into the pericardial space. Such infusion could be directed towards the epicardial surface, if so desired, by introducing porosity only along the side 82a of the outer balloon 80 facing the epicardium.

As an alternative to a double balloon 80, in a single balloon construction, the everting balloon 20 itself can be porous. Drug solution can be placed in the chamber 24 and pass across the material of the everting balloon 20 when pressurized. Pore size and density can be controlled to give the desired flow characteristics. Preferably, the material of the balloon is microporous to produce a relatively slow release of drug solution.

Blood Control from Atrium to Pericardial Space

It may not be necessary to control passage of blood from the right atrium to the pericardial space, during deployment of the passive constraint device. Factors such as atrial blood pressure, pericardial fluid pressure, size of access hole across the atrium, and location of the access hole can influence the propensity for blood to cross into the pericardial space. As previously mentioned, a suture can be applied to the access hole.

The device-introduction catheter can carry a suction lumen to enable fluid within the pericardial space to be evacuated by syringe, attached to a proximal end of the device-introduction catheter. As noted in the afore-mentioned Verrier et al article, it may not be necessary to perform any type of repair process on the transatrial access site. When the device-introduction catheter and/or guide catheter are withdrawn away from the transatrial access site, the site will likely promote localized thrombus formation. If a repair of the site should be necessary, this may be accomplished by attaching a clip, or inflating a double-button device across the transatrial access site. Alternatively, methods such as are used to promote hemostasis at the site of introducer access to peripheral blood vessels may be used. Also, a protein plug, consisting of collagen or other procoagulant protein may be inserted into the transatrial access penetration site.

Magnetic Guidance

As previously discussed, magnetic guidance can be used as a means of directing or positioning the passive constraint device or guide member. Such positioning capability can be used not only for positioning a passive constraint device but also for positioning other therapeutic implements. One example includes a catheter extension incorporating an injection needle intended for direct injection of bioactive agents, including small molecular weight pharmaceutical agents, genes, gene products (proteins, mRNAs), gene product antagonists or agonists (such as antisense oligonucleotides or small interfering RNAs (siRNA), or cells, across the epicardium and into the myocardium. A particularly attractive approach would be to use a steering system to direct the injection site for introduction of cells intended to integrate into, and improve contractile performance of the myocardium. Alternative bioactive agents include those know in the art for influencing survival and integration of cell transplants. Such agents can also include genes or gene products for growth factors and cytokines, among others.

In addition to magnetic guidance, other approaches to positioning the passive constraint device, or other therapeutic implements can be used. In one case, instead of the passive constraint device consisting of a shape memory material such as nitinol (as previously described), deployed from within an everting balloon, the passive constraint device can have the ability to bend or torque by mechanical means, under direction of the operator while viewing a fluoroscopic image in such a way that the passive constraint device can subtly alter orientation of the everting balloon as it extends around the heart in a circumferential or helical pattern. Such ability to alter the course of travel as the passive constraint device is deployed would be conferred upon the device by means known in the art (e.g., mechanical structures such as those used in bendable or steer-able endoscopes and the like). For example, U.S. patent application publication No. 2004/0236316 A1 published Nov. 25, 2004 teaches an articulating mechanism for remote manipulation of a surgical or diagnostic tool. Similarly, such a tool is shown in International Publication No. WO 2004/105578 A2 published Dec. 9, 2004 and assigned to Novare Surgical Systems, Inc., Cupertino, Calif., USA.

Alternatively, torque or bending can be exerted on the central everting balloon/passive constraint device construct by outrider tensioning members also deployed with the everting balloon/passive constraint device (e.g., similar to those shown in FIG. 4C for deploying a device that is relatively flat in cross-sectional profile). The outrider tensioning members could be deployed in conjunction with the device-introduction catheter. Tensioning of the outrider members would then be used to alter the orientation or course of deployment of the passive constraint device. The means of tensioning the outrider can include tensioning of a wire insert, or due to inflation pressure within an outrider balloon.

Having disclosed the invention of preferred embodiment, it will be appreciated that modifications and equivalents of the disclosed concepts may occur to one of ordinary skill in the art having the benefit of the teachings of the present invention. It is intended that such modifications and equivalents shall be included within the scope of the appended claims.

We claim:

1. A method for treating a disease of a heart comprising:
forming an access opening from a heart chamber into a pericardial space defined between an epicardial surface of the heart and a pericardium opposing said epicardial surface;
deploying a cardiac support member into said pericardial space through said access opening with said cardiac support member selected to engage an epicardial surface of said heart and relieve a wall tension of said heart.

2. A method according to claim 1 further comprising selecting a support member having a pre-formed shape selected to at least partial surround said heart after said deployment.

3. A method according to claim 1 further comprising admitting a guide member into said pericardial space through said access opening and positioning said guide member in a desired disposition for said cardiac support member; said deployment of said cardiac support member including advancing said cardiac support member to said desired position after said deployment of said guide member with said guide member guiding said cardiac support member to said desired position.

4. A method according to claim 3 wherein said guide member is a guide wire and said cardiac support member includes a lumen for slidably receiving said guide wire.

5. A method according to claim 3 wherein said guide member is an everting member everted during said deployment to said desired disposition.

6. A method according to claim 1 wherein said cardiac support member is formed at least in part from a polymer.

7. A method according to claim 6 wherein said polymer is selected to induce a tissue response from said epicardial surface.

8. A method according to claim 1 wherein said cardiac support member is formed at least in part from a shape-memory alloy.

9. A method according to claim 1 wherein said cardiac support member includes a plurality of tissue attachment locations along a length thereof.

10. A method according to claim 9 wherein said tissue attachment locations include a bio-adhesive.

11. A method according to claim 9 wherein said tissue attachment locations include a member for piercing said epicardial surface.

12. A method according to claim 9 wherein said tissue attachment locations include a material selected to induce a fibrotic response from said epicardium.

13. A method according to claim 1 wherein said cardiac support member includes a plurality of electrodes along a length thereof and disposed to oppose said epicardial surface.

14. A method according to claim 1 wherein said cardiac support member includes a spacer placed within said pericardial space and said method includes treating a portion of said pericardium opposing said spacer with a treatment selected to stiffen said portion.

15. An apparatus for treating a disease of the heart comprising:
a cardiac support member sized and configured to be passed through an atrial wall of a human heart and reside within a pericardial space defined between opposing surfaces of a pericardium and an epicardium;
said cardiac support member adapted to be positioned within said pericardial space and opposing a non-resisted diastolic expansion of said heart.

16. An apparatus according to claim 15 comprising an everting member sized to be introduced into said pericardial space and be everted within said space to a pre-formed configuration surrounding at least a portion of said heart.

17. A kit for treating a disease of the heart comprising;
an introducer for accessing a pericardial space defined between opposing surfaces of a pericardium and an epicardium by passing through atrial wall of a human heart;
a cardiac support member sized and configured to be passed through said introducer into said atrial wall and be positioned within said pericardial space and opposing a non-resisted diastolic expansion of said heart.

18. A kit according to claim 17 further comprising:
a guide wire sized and configured to be passed through said introducer into said atrial wall and be positioned within said pericardial space in a desired pattern;
said cardiac support member adapted to be urged into said pericardial space while being guided by said guide wire to said desired pattern.

19. A kit according to claim 17 wherein said introducer is a catheter adapted for percutaneous delivery to an atrium of said heart.

20. A kit according to claim 17 wherein said cardiac support member is an everting member sized to be introduced into said pericardial space and be everted within said space to a pre-formed configuration surrounding at least a portion of said heart.
